(19) United States
(12) Patent Application Publication
(10) Pub. No.: US 2007/0225584 A1
(43) Pub. Date: Sep. 27, 2007

(54) Novel Catheter Sensor

(75) Inventors: Nikolaus Gravenstein, Gainesville, FL (US); Tammy Y. Euliano, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Florida ResearouchFoundation, Inc., Gainesville, FL (US)

(21) Appl. No.: 11/547,459

(22) PCT Filed: Apr. 18, 2005
(86) PCT No.: PCT/US05/13092

§ 371 Date: Oct. 4, 2006

Related U.S. Application Data
(60) Provisional application No. 60/563,846, filed on Apr. 19, 2004.

(51) Int. Cl. A61B 5/042

(52) U.S. Cl. 600/376

(57) ABSTRACT

A fetal monitoring device directed to a maternal bladder insert having at least one sensor on the distal end to detect fetal vital signs and uterine activity, and methods for detecting fetal vital signs and uterine activity using the device. The bladder insert is preferably a catheter with an integrated electrode for detecting fetal heart rate and uterine electromyography. Furthermore, the device transmits this data to a monitoring system for diagnosis and observation.

### GOVERNMENT SUPPORT

This invention was made with government support under a grant awarded from the National Science Foundation under grant number 023960. The government has certain rights in the invention.

### BACKGROUND OF INVENTION

This invention is directed to a device and method for detecting a fetus's heart rate and ECG, as well as maternal heart rate and uterine contraction pattern/strength when the device is inserted into the maternal bladder.

Assessment of the fetus during pregnancy, and particularly during labor and delivery, is an essential but yet elusive goal. While most patients will deliver a healthy child with or without monitoring, more than 5 out of every 1,000 deliveries of a viable fetus near term is stillborn, with half having an undetermined cause of death. (National Vital Statistics System (NVSS), CDC, NCHS as published in “Healthy People 2010, Understanding and Improving Health: Chapter 16,” co-authored by the Centers for Disease Control and Prevention and Health Resources and Services Administration, 2^{nd} Edition, U.S. Government Printing Office, November 2000).

Intrapartum fetal surveillance routinely consists of intermittent auscultation or continuous Doppler monitoring of the fetal heart rate (FHR), together with palpation or tocodynamometry (strain gauge) monitoring of contractions. When indicated, more invasive monitors are available, but require ruptured membranes/adequate cervical dilation, and entail some risk, primarily infectious. These monitors include, without limitation:
1. fetal scalp electrode—a wire electrode inserted into the fetal scalp;
2. intra-uterine pressure catheter (IUPC)—enables quantitative measurement of contractions; and
3. fetal scalp sampling—a blood sample drawn for pH analysis.

Furthermore, during non-obstetric surgery in the pregnant patient, monitoring of the fetus can be difficult or impossible, depending on the location of the surgery (e.g. abdominal surgery where the monitoring Doppler unit would be in the way in the sterile field) and gestational age of the fetus. Even if the fetus is “pre-viable” (<24 weeks gestation), knowledge of fetal ischemia could alter the management of the patient, to improve the intra-uterine environment (e.g., increase oxygen supply or blood pressure). Similarly, the greatest risk is preterm delivery following non-obstetric surgery in the pregnant patient, yet contractions are not routinely monitored in part due to the complexity of the equipment and frequent lack of access to the desired site (e.g., abdominal dressing).

During labor, progress is determined by serial cervical examinations. In the interim, the contraction monitor displays the pattern of uterine contractions. The non-invasive tocodynamometer detects only the presence or absence of tension on the abdomen (whether from uterine contraction or maternal movement), and often fails in the obese patient. When cervical dilation lags behind the anticipated labor curve, oxytocin is often indicated to induce a more effective contraction pattern. Safe titration of the oxytocin may require accurate determination of “Montevideo units” which measure the strength of uterine contractions over 10 minutes. This requires the more invasive IUPC, a catheter placed into the uterus, alongside the fetus, to measure the pressure generated by uterine contractions.

In addition to monitoring the contraction pattern, the rationale for use of intrapartum electronic fetal monitoring (EFM) assumes that FHR abnormalities accurately reflect hypoxia (inadequate oxygen to the fetus), and that early recognition of this could induce intervention to improve outcome for both mother and fetus. Unfortunately, numerous studies have failed to realize this improved outcome with the use of EFM in low-risk deliveries. In fact some studies have actually shown an increase in morbidity from a higher operative delivery rate. Perhaps this should not be surprising in light of the variability in interpretation of FHR tracings and their lack of specificity for hypoxia. Yet, continuous EFM remains the standard of care in US hospitals, in large part due to medicolegal concerns. Meanwhile researchers seek an alternative monitor, specific for fetal well being, preferably one that is non-invasive and comfortable for the mother, with reliable, reproducible interpretation.

Recently, analysis of the fetal electrocardiogram (FECG) has held promise, with some features of the waveform more specifically indicating fetal hypoxia. Use of the waveform analysis reduced the incidence of severe metabolic acidosis at birth, while necessitating fewer scalp samples and operative deliveries. Unfortunately, acquisition of the FECG was through the fetal scalp electrode described above which is both invasive and limited in its application. The necessity for access to the fetal scalp requires both adequate cervical dilation and ruptured membranes, eliminating this procedure for antepartum fetal surveillance, as well as early labor.

Devices that utilize invasive techniques for monitoring fetal health include those disclosed in U.S. Pat. Nos. 6,594,515; 6,115,624; 6,058,321; 5,746,212; 5,184,619; 4,951,680; and 4,437,467.

To address the inadequacies noted above, various methods have been proposed for use in processing maternal abdominal signals to provide more accurate FECG extraction. These methods include subtractive filtering (see, for example, U.S. Pat. No. 4,945,917), adaptive filtering (see, for example, Widrow, B. et al., “Adaptive Noise Canceling: Principals and Applications,” Proc. IEEE, 63(12):1692-1716 (December 1975); Adam, D. and D. Shavit, “Complete Fetal ECG Morphology Recording by Synchronized Adaptive Filtration,” Med. & Biol. Eng. & Comput., 28:287-292 (July 1990); Ferrara, E. and B. Widrow, “Fetal Electrocardiogram Enhancement by Time Sequenced Adaptive Filtering,” IEEE Trans. Biomed. Eng., BME-29(6):458-460 (June 1982); U.S. Pat. Nos. 4,781,200 and 5,042,499), orthogonal basis (Longini, R. et al., “Near Orthogonal Basis Function: A Real Time Fetal ECG Technique,” IEEE Trans. On Biomedical Eng., BME-24(1):39-43 (January 1977); U.S. Pat. No. 5,042,499), linear combination (Bergveld, P. et al., “Real Time Fetal ECG Recording,” IEEE Trans. On Beiomedical Eng., BME-33(5):505-509 (May 1986)), single value decomposition (Callaerts, D. et al., “Comparison of SVD Methods to Extract the Fetal Electrocardiograrn from Cutaneous Electrodes Signals,” Med. & Biol. Eng. & Comput., 28:217-224 (May 1990); U.S. Pat. No. 5,209,237), and MECG averaging and correlation (Abboud, S. et al., “Quantification of the Fetal Electrocardiogram Using Averaging Technique,” Comput. Biol. Med., 20:147-155 (February 1990); Cerutti, S. et al., “Variability Analysis of Fetal Heart Rate Signals as Obtained from Abdominal Electrocardiographic Recordings,” J. Perinat. Med., 14:445-452 (1986); J. Nagel, “Progresses in Fetal Monitoring by Improved Data Acquisition,” IEEE Eng. Med. & Biol. Mag., 9-13 (September 1984); Oostendorp, T. et al., “The Potential Distribution Generated by Fetal Heart at the Maternal Abdomen,” J. Perinat. Med., 14:435-444 (1986); U.S. Pat. No. 5,490,515). These methods, unfortunately, do not enable real-time extraction of maternal-fetal data or cannot capture a comprehensive account of maternal-fetal health based on a combination of test results (i.e., fetal heart rate, fetal ECG, maternal ECG, and maternal uterine activity (EHG)).

Recently, magnetocardiography has been utilized in extracting FECG (see, for example, Sturm, R. et al., “Multi-channel magnetocardiography for detecting beat morphology variations in fetal arrhythmias,” Prenat Diagn, 24(1):1-9 (January 2004); and Stinstra, J. et al., “Multicentre study of fetal cardiac time intervals using magnetocardiography,” BJOG, 109(11):1235-43 (November 2002)). Unfortunately, magnetocardiography is limited in application, technologically complex, and difficult to administer to assess accurate fetal ECG readings.

Accordingly, a device that measures FECG with low risk to the fetus is needed that can monitor in real time FECG intrapartum and antepartum.

### BRIEF SUMMARY OF THE INVENTION

It is an object of the subject invention to provide a device and methods for monitoring fetal health and labor quality. More particularly, it is an object to provide a device and methods for detecting FECG and maternal uterine electromyogram (electrohysterogram, EHG).

It is a further object of the subject invention to provide a bladder device and methods for monitoring FECG antepartum and/or intrapartum. It is also an object to provide a less invasive monitoring device and methods.

It is a further object of the subject invention to provide a way to transmit the detected vital signs to a monitoring system external to the mother. The monitoring system allows the attending physician and/or medical staff to observe and diagnose any issues related with the pregnancy, including both maternal and fetal health.

### BRIEF DESCRIPTION OF DRAWINGS

FIG. 1 illustrates a fetal monitoring device of the subject invention, including a bladder insert, an electrophysical sensor, and a means for transmitting extracted fetal vital signals from the sensor to a maternal fetal monitoring system.

FIG. 2 illustrates a bladder insert having a retention bladder with an electrophysical sensor on the distal end and an electrophysical sensor on the retention bladder.

### DETAILED DISCLOSURE

One aspect of the subject invention is directed to devices useful for monitoring fetal vital signs while the device is inserted into the maternal bladder. Advantageously, the fetal monitoring device of the invention extracts vital signs, specifically FECG in real-time using electrophysical sensors. The sensor location is external to the uterus, and thus has no requirement for cervical dilation or membrane rupture. A further advantage lies in a reduced risk of danger to the fetus because the monitoring device of the subject invention and the fetus are not in direct contact. Furthermore, the device of the invention is useful antepartum and intrapartum because the device is designed for insertion into the maternal bladder.

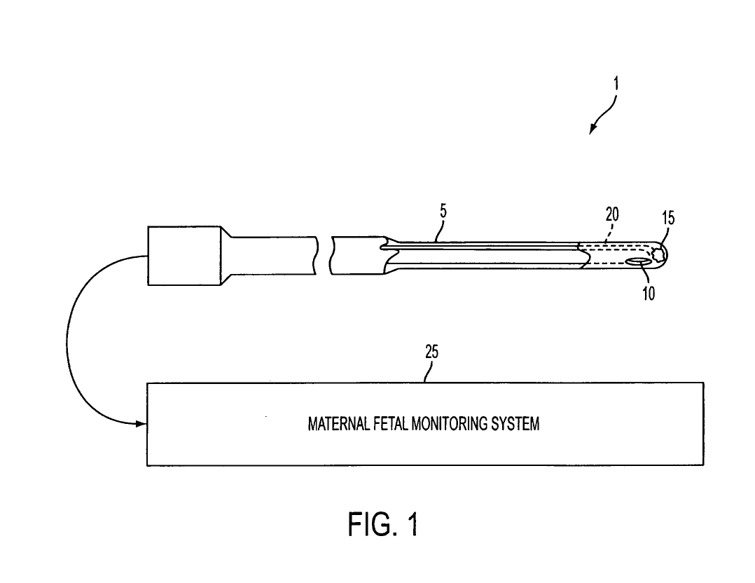

The fetal monitoring device of the subject invention comprises a bladder insert, at least one electrophysical sensor, and a means to transmit extracted fetal vital signs from the sensor to a maternal fetal monitoring system external to the patient. In FIG. 1, a fetal monitoring device 1 of the subject invention is illustrated, wherein a bladder insert 5 is provided having one or more inlets 10 for urine drainage. One or more electrophysical sensors 15 are either integrally formed on or inserted at the distal portion of the bladder insert of the subject invention or to be inserted into a commercially available bladder catheter. Additional electrodes may be positioned on the skin as well. A lead 20 is connected to the sensor to provide a means for transmitting extracted fetal vital signals from the sensor 15 to a maternal fetal monitoring system 25.

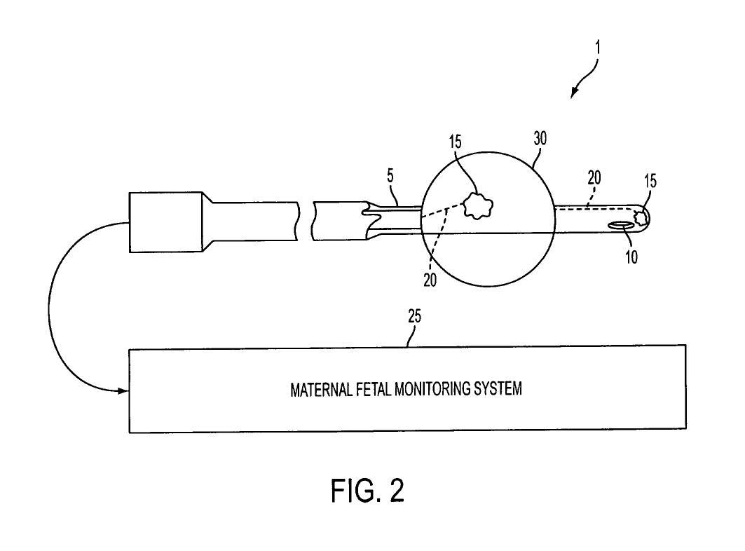

In FIG. 2, one embodiment of the invention is illustrated wherein a fetal monitoring device 1 includes a bladder insert 5 having one or more inlets 10 for urine drainage and a retention balloon 30. One or more electrophysical sensors 15 are either integrally formed on or inserted at the distal portion of the bladder insert 5 and/or on the retention balloon 30 (either of the subject invention or to be inserted into a commercially available bladder catheter), additional electrodes may be positioned on the skin as well. A lead 20 is connected to the sensor to provide a means for transmitting extracted fetal vital signals from the sensor 15 to a maternal fetal monitoring system 25.

As used herein, the term “vital signs” or “vital signals” includes maternal and fetal heart rate, respiratory rate, ECG results, and EHG.

As used herein, the term “clinical data” refers to information obtained from the analysis and/or interpretation of maternal-fetal vital signs. Clinical data can include, but is not limited to, classification of maternal and fetal health (i.e., normal fetal heart rate or normal maternal heart rate during labor), fetal presentation, labor progress, contraction efficiency, pharmaceutical efficacy, arrhythmias, bradycardia, tachycardia, and problems with umbilical cord or with fetal presentation, also problems with uterine contractions or uterine rupture.

As used herein, the term “patient” refers to a mother and/or fetus. The term patient includes mammals to which monitoring systems according to the subject invention are provided. Mammalian species that benefit from the disclosed monitoring systems include, but are not limited to, domesticated animals or rare animals that require observation in the zoo or wild.

As used herein, the term “catheter guide” refers to a flexible metallic wire or thin sound over which a catheter is passed to advance it into its proper position.

The bladder insert of the subject invention comprises a flexible tubular device having a distal end and a proximal end. Preferably, the bladder insert is a catheter for insertion into a maternal bladder. More preferably, the bladder insert is a catheter for insertion into a maternal bladder, wherein the catheter includes a Foley retention balloon which in use is situated within the bladder to prevent inadvertent removal of the catheter. According to the subject invention, with certain embodiments, the bladder insert can be manufactured with an electrophysical sensor integrated into the structure of the bladder insert and/or on to the external surface of the retention balloon. Preferably, a sensor is located on the equator of the retention balloon. Alternatively, an electrophysical sensor of the invention can be inserted into a conventional catheter used for insertion into a bladder.

The bladder insert is constructed of a material that is both flexible and substantially strong enough to support at least one sensor at its distal end. Preferably, the bladder insert is constructed of latex, Teflon, or silicon rubber. The device of the subject invention is available in various sizes, for example, 3 to 7 French.

The fetal monitoring device of the subject invention optionally comprises a catheter guide means for positioning the bladder insert into the maternal bladder.

The electrophysical sensors of the subject invention monitor the electrical impulses arriving at the sensor location, including those generated by the fetus and the mother, including the uterus. In one embodiment, a single sensor is integrated into the distal end of the bladder insert. In another embodiment, a single sensor is attached to a lead that is attached to the distal end of the bladder insert. In yet another embodiment, a plurality of sensors are attached to a plurality of leads. In yet another embodiment, a plurality of sensors are attached to a first plurality of leads, and a second plurality of sensors attached to a second plurality of leads spaced apart from the first plurality of leads. These leads and sensors may be internal and/or combined with external sensors and leads. In yet another embodiment, a sensor is attached to the outer surface of the retention balloon.

In a preferred embodiment, the sensor is an electrode that is integrated into a catheter (i.e., bladder catheter). For example, electrode-integrated catheters such as those disclosed in U.S. Pat. Nos. 6,682,526; 6,610,054; and 5,697,927 can be used in the maternal-fetal monitoring system of the subject invention.

In accordance with the device of the subject invention, a plurality of electrodes is attached, using multiple leads, to the distal end of the bladder insert.

The transmitting means of the subject invention allows the detected vital signs to be transferred to an external maternal-fetal monitoring system. The transmitting means can include, without limitation, a transmitter equipped with wireless medical telemetry technology and a cable connection attaching the device and the monitoring system. The transmitting means optionally includes transducers, filters, amplifiers, analog to digital converters or any other signal that readies the FECG signal for further processing. In one embodiment, FECG signals are converted into a heart rate that is recorded onto a printer, for example, a strip chart.

In another embodiment, FECG obtained in accordance with the subject invention are transmitted from the sensors to a computing means for signal processing. The computing means can also be responsible for maintenance of acquired data as well as the maintenance of the maternal-fetal monitoring system itself. The computing means can also detect and act upon user input via user interface means known to the skilled artisan (i.e., keyboard, interactive graphical monitors).

In one embodiment, the computing means further comprises means for storing and means for outputting processed data. The computing means includes any digital instrumentation capable of processing signals communicated from the sensor of the invention (i.e., ECG signals). Such digital instrumentation, as understood by the skilled artisan, can process communicated signals by applying algorithm and filter operations of the subject invention. Preferably, the digital instrumentation is a microprocessor, a personal desktop computer, a laptop, and/or a portable palm device. The computing means can be general purpose or application-specific.

The fetal monitoring device of the subject invention further comprises a power source. Preferably, the power supply includes an adapter that is a 12V AC-DC medical grade power supply adapter and a power converter, which is provided to protect the patient from leakage currents. Alternatively, the power source is a rechargeable or replaceable battery. Preferably, the battery source is a lithium battery.

Another aspect of the subject invention is directed to methods for monitoring fetal electrocardiogram from the maternal bladder.

One preferred method is directed to inserting a fetal monitoring device, in accordance with the subject invention, into the bladder of the mother, detecting the electrical impulses generated by the fetal heartbeat, and transmitting the impulses to a fetal monitoring system.

Insertion requires passing the fetal monitoring device upwardly through the maternal urethra into the bladder. Insertion of the catheter is accomplished using techniques known to the skilled artisan. In a related embodiment, the insertion step can include the use a catheter guide to aid in the insertion of the fetal monitoring device of the subject invention in the maternal bladder.

The detection step comprises supplying power to the device of the subject invention and capturing FECG signals. In certain embodiments, the location of the bladder insert of the subject invention can be monitored and adjusted for optimal detection and capture of FECG signals. Alternatively, the location of the electrophysical sensor can be monitored and adjusted for optimal detection and capture of FECG signals.

The transmitting step is directed to transferring captured FECG signals to an external maternal-fetal monitoring system. The transmitting step utilizes principles of medical telemetry to transfer any captured raw data.

In one embodiment, a transmitter equipped with wireless medical telemetry technology is connected to the electrodes in conjunction with the device of the subject invention. Advantageously, the transmitter is worn by the patient, which allows freedom of movement. In addition, wireless transmitting allows remote observation and diagnosis of the patient.

In another embodiment, the detected signals are transmitted via a hard wire connection to an external maternal-fetal monitoring system. This embodiment is particularly useful when the patient is on bed rest, or undergoing non-obstetric surgery during pregnancy.

The external maternal-fetal monitoring system can include, without limitation, computing means for processing the transmitted signal and converting the signals to an output format, for example, printed on a strip chart.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A catheter useful for monitoring fetal vital signs from a maternal bladder, wherein the catheter comprises:
a) a bladder insert comprising a distal end and a proximal end and at least one inlet useful for urine drainage when in use;
b) at least one electrophysical sensor; and
c) a means for transmitting data collected by the at least one electrophysical sensor.

2. The catheter according to claim 1, further comprising:
d) a retention balloon interposed between the distal end of the bladder insert and the proximal end of the bladder insert.

3. The catheter according to claim 2, further comprising:
e) at least one lead attached to the distal end of the bladder insert.

4. The catheter according to claim 1, further comprising:
f) at least one lead attached to the distal end of the bladder insert.

5. The catheter according to claim 2, wherein the at least one electrophysical sensor is integrated onto the surface of the retention balloon.

6. The catheter according to claim, wherein the at least one electrophysical sensor is integrated onto the surface of the retention balloon at its equator.

7. The catheter according to claim 1, wherein the at least one electrophysical sensor is integrated into the structure of the bladder insert.

8. The catheter according to claim 1, wherein one electrophysical sensor is integrated into the distal end of the bladder insert.

9. The catheter according to claim 3, wherein one electrophysical sensor is attached to one lead.

10. The catheter according to claim 3, comprising at least two electrophysical sensors and at least two leads; wherein one electrophysical sensor is attached to one of said leads.

11. The catheter according to claim 10, wherein each of the at least two leads are uniformly spaced apart from each other.

12. The catheter according to claim 3, wherein the at least one lead comprises a first plurality of leads attached to a first plurality of electrophysical sensors and a second plurality of leads attached to a second plurality of electrophysical sensors; and wherein the first plurality of leads are spaced apart from the second plurality of leads.

13. The catheter according to claim 1, wherein one electrophysical sensor is an electrode.

14. The catheter according to claim 1, wherein the transmitting means is a wireless transmitter.

15. The catheter according to claim 1, wherein the transmitting means is a cable connection between the bladder insert and a computing means external to the bladder insert.

16. The catheter according to claim 1, wherein the bladder insert comprises a flexible material selected to bear the weight of at least one electrophysical sensor without degrading.

17. The catheter according to claim 1, wherein the bladder insert comprises latex, polytetrafluoroethylene, silicon rubber, or a combination of the foregoing.

18. A fetal monitoring device useful for monitoring antepartum and intrapartum fetal electrocardiogram signals comprising:
a) a catheter comprising:
i) a bladder insert comprising a distal end and a proximal end and at least one inlet useful for urine drainage when in use, at least one electrophysical sensor, and a means for transmitting data collected by the at least one electrophysical sensors;
ii) a bladder insert comprising a distal end and a proximal end and at least one inlet useful for urine drainage when in use, at least one electrophysical sensor, a means for transmitting data collected by the at least one electrophysical sensors, and a retention balloon interposed between the distal end of the bladder insert and the proximal end of the bladder insert;
iii) a bladder insert comprising a distal end and a proximal end and at least one inlet useful for urine drainage when in use, at least one electrophysical sensor, a means for transmitting data collected by the at least one electrophysical sensors, a retention balloon interposed between the distal end of the bladder insert and the proximal end of the bladder insert, and at least one lead attached to the distal end of the bladder insert; or
iv) a bladder insert comprising a distal end and a proximal end and at least one inlet useful for urine drainage when in use, at least one electrophysical sensor, a means for transmitting data collected by the at least one electrophysical sensors, and at least one lead attached to the distal end of the bladder insert; and
b) a maternal fetal monitoring system, wherein the maternal fetal monitoring system is external to a maternal patient when in use.

19. The fetal monitoring device according to claim 18, further comprising:
c) at least one electrode that can be positioned on the skin of the maternal patient.

20. The fetal monitoring device according to claim 18, further comprising:
d) at least one electrode that can be positioned on the skin of the maternal patient; and
e) a power source.

21. The fetal monitoring device according to claim 18, further comprising:
f) a power source.

22. The fetal monitoring device according to claim 18, further comprising:
g) at least one electrode that can be positioned on the skin of the maternal patient;
h) a power source; and
i) a means for guiding the catheter into a bladder of the maternal patient.

23. The fetal monitoring device according to claim 18, further comprising:
j) a means for guiding the catheter into a bladder of the maternal patient.

24. The fetal monitoring device according to claim 19, further comprising:
k) a means for guiding the catheter into a bladder of the maternal patient.

25. The fetal monitoring device according to claim 21, further comprising:
l) a means for guiding the catheter into a bladder of the maternal patient.

26-29. (canceled)

30. The fetal monitoring device according to claim 18, wherein the fetal monitoring system comprises a means for processing fetal electrocardiogram signals and a means for converting fetal electrocardiogram signals into output.

31. The fetal monitoring device according to claim 18, wherein the at least one electrophysical sensor is integrated onto the surface of the retention balloon.

32. The fetal monitoring device according to claim 18, wherein the at least one electrophysical sensor is integrated into the surface of the retention balloon at its equator.

33. The fetal monitoring device according to claim 18, wherein the at least one electrophysical sensor is integrated into the structure of the bladder insert.

34. The fetal monitoring device according to claim 18, wherein one electrophysical sensor is integrated into the distal end of the bladder insert.

35. The fetal monitoring device according to claim 18, wherein one electrophysical sensor is attached to one lead.

36. The fetal monitoring device according to claim 18, comprising at least two electrophysical sensors and at least two leads; wherein one electrophysical sensor is attached to one of said leads.

37. The fetal monitoring device according to claim 18, wherein each of the at least two leads are uniformly spaced apart from each other.

38. The fetal monitoring device according to claim 18, wherein the at least one electrophysical sensors comprise a first plurality of electrophysical sensors and a second plurality of electrophysical sensors; wherein the at least one lead comprises a first plurality of leads and a second plurality of leads, wherein the first plurality of electrophysical sensors are attached to the first plurality of leads; wherein the second plurality of electrophysical sensors are attached to the second plurality of leads; and wherein the first plurality of leads are spaced apart from the second plurality of leads.

39. The fetal monitoring device according to claim 18, wherein one electrophysical sensor is an electrode.

40. The fetal monitoring device according to claim 18, wherein the transmitting means is a wireless transmitter.

41. The fetal monitoring device according to claim 18, wherein the transmitting means is a cable connection between the bladder insert and a computing means external to the bladder insert.

42. The fetal monitoring device according to claim 18, wherein the bladder insert comprises a flexible material selected to bear the weight of at least one electrophysical sensor without degrading.

43. The fetal monitoring device according to claim 18, wherein the bladder insert comprises latex, polytetrafluoroethylene, silicon rubber, or a combination of the foregoing.

44. A method for collecting fetal vital signs comprising:
a) inserting a catheter into the bladder of a maternal patient, wherein the catheter comprises:
i) a bladder insert comprising a distal end and a proximal end and at least one inlet useful for urine drainage when in use, at least one electrophysical sensor, and a means for transmitting data collected by the at least one electrophysical sensors;
ii) a bladder insert comprising a distal end and a proximal end and at least one inlet useful for urine drainage when in use, at least one electrophysical sensor, a means for transmitting data collected by the at least one electrophysical sensors, and a retention balloon interposed between the distal end of the bladder insert and the proximal end of the bladder insert;
iii) a bladder insert comprising a distal end and a proximal end and at least one inlet useful for urine drainage when in use, at least one electrophysical sensor, a means for transmitting data collected by the at least one electrophysical sensors, a retention balloon interposed between the distal end of the bladder insert and the proximal end of the bladder insert, and at least one lead attached to the distal end of the bladder insert; or
iv) a bladder insert comprising a distal end and a proximal end and at least one inlet useful for urine drainage when in use, at least one electrophysical sensor, a means for transmitting data collected by the at least one electrophysical sensors, and at least one lead attached to the distal end of the bladder insert;
b) detecting electric impulses generated by a fetal heartbeat; and
c) transmitting the electric impulses to a fetal monitoring system.

45. The method according to claim 44, wherein the inserting step comprises using a catheter guide.

46. The method according to claim 44, wherein the detecting step comprises supplying power to the fetal monitoring device and capturing FECG signals.

47. The method according to claim 44, wherein the detecting step comprises adjusting the location of the at least one electrophysical sensors.

48. The method according to claim 44, wherein the at least one electrophysical sensor is integrated onto the surface of the retention bladder.

49. The method according to claim 44, wherein the at least one electrophysical sensor is integrated into the surface of the retention balloon at its equator.

50. The method according to claim 44, wherein the at least one electrophysical sensor is integrated into the structure of the bladder insert.

51. The method according to claim 44, wherein one electrophysical sensor is integrated into the distal end of the bladder insert.

52. The method according to claim 44, wherein one electrophysical sensor is attached to one lead.

53. The method according to claim 44, comprising at least two electrophysical sensors and at least two leads; wherein each of the at least two electrophysical sensors are individually attached to each of the at least two leads.

54. The method according to claim 44, wherein each of the at least two leads are uniformly spaced apart from each other.

55. The method according to claim 44, wherein the at least one electrophysical sensors comprise a first plurality of electrophysical sensors and a second plurality of electrophysical sensors; wherein the at least one lead comprises a first plurality of leads and a second plurality of leads, wherein the first plurality of electrophysical sensors are attached to the first plurality of leads; wherein the second plurality of electrophysical sensors are attached to the second plurality of leads; and wherein the first plurality of leads are spaced apart from the second plurality of leads.

56. The method according to claim 44, wherein one electrophysical sensor is an electrode.

57. The method according to claim 44, wherein the transmitting means is a wireless transmitter.

58. The method according to claim 44, wherein the transmitting means is a cable connection between the bladder insert and a computing means external to the bladder insert.

59. The method according to claim 44, wherein the bladder insert comprises a flexible material selected to bear the weight of at least one electrophysical sensor without degrading.

60. The method according to claim 44, wherein the bladder insert comprises latex, polytetrafluoroethylene, silicon rubber, or a combination of the foregoing.
